(19) United States
(12) Patent Application Publication
(10) Pub. No.: US 2026/0117318 A1
(43) Pub. Date: Apr. 30, 2026

(54) REAL-TIME PCR ASSAY FOR SIMULTANEOUSLY DETECTING MYCOPLASMA GENITALIUM AND ITS MUTATIONS

(71) Applicant: THE UAB RESEARCH FOUNDATION, BIRMINGHAM, AL (US)

(72) Inventors: Li XIAO, BIRMINGHAM, AL (US); Amy E. RATLIFF, BIRMINGHAM, AL (US); Hong WANG, BIRMINGHAM, AL (US); Ken B. WAITES, BIRMINGHAM, AL (US); William Michael GEISLER, BIRMINGHAM, AL (US)

(21) Appl. No.: 19/373,026

(22) Filed: Oct. 29, 2025

Related U.S. Application Data
(60) Provisional application No. 63/713,308, filed on Oct. 29, 2024.

(51) Int. Cl. C12Q 1/689; C12Q 1/6818; C12Q 1/686

(52) U.S. Cl. CPC C12Q 1/689; C12Q 1/6818; C12Q 1/686; C12Q 2600/156

(57) ABSTRACT

Described herein are primers, probes, kits, compositions, and methods related to detection of Mycoplasma genitalium (MG) and mutations in quinolone resistance determining region (QRDR) of parC and gyrA.

### CROSS-REFERENCE TO RELATED APPLICATION[S]

This application claims priority to, and the benefit of, U.S. Provisional Application No. 63/713,308, filed on Oct. 29, 2024, the entire contents of which are incorporated by reference as if fully set forth herein.

### SEQUENCE LISTING

The instant application contains a Sequence Listing which has been submitted electronically in .XML format and is hereby incorporated by reference in its entirety. Said .XML copy, created on Oct. 29, 2025, is named “222120_1030_sequence_listing.xml” and is 11,686 bytes in size.

### BACKGROUND

Mycoplasma genitalium (MG) causes urethritis in men and cervicitis in women, and is associated with pelvic inflammatory disease, preterm delivery, spontaneous abortion, and infertility in women. MG lacks a cell wall and is the smallest bacterium capable of independent replication (genome size 0.58 Mbp). MG is extremely slow-growing, and culture takes up to 6 months.

CDC Guidelines Recommendation for MG Testing and Treatment include MG testing with a nucleic acid amplification test (NAAT) for those with recurrent/persistent urethritis or cervicitis, and women with PID, and then sequential treatment with doxycycline followed by azithromycin or moxifloxacin depending on resistance status. There is no FDA-cleared MG NAATs include detection of MG resistance.

Macrolide and fluoroquinolone resistance is increasing in the US: Macrolide Resistance: 31% to 94%. No longer a good option for MG treatment. Fluoroquinolone Resistance: 6% to 30%. There is a need for a resistance test to coordinate MG treatment strategies accordingly.

### SUMMARY

Described herein are primers, probes, kits, compositions, and methods related to detection of Mycoplasma genitalium (MG) and mutations in quinolone resistance determining region (QRDR) of parC and gyrA.

In embodiments, described herein are donor-acceptor fluorescence resonance energy transfer (FRET) probe pairs. In embodiments, a parC donor-acceptor pair can comprise a donor probe having at least 90% sequence identity to SEQ ID NO: 3 and an acceptor probe having at least 90% sequence identity to SEQ ID NO: 4. In embodiments, a gyrA donor-acceptor pair can comprise a donor probe having at least 90% sequence identity to SEQ ID NO: 7 and an acceptor probe having at least 90% sequence identity to SEQ ID NO: 8.

In embodiments, the parC donor-acceptor pair can comprise a donor probe having at least 95% sequence identity to SEQ ID NO: 3 and an acceptor probe having at least 95% sequence identity to SEQ ID NO: 4. In embodiments, the gyrA donor-acceptor pair can comprise a donor probe having at least 95% sequence identity to SEQ ID NO: 7 and an acceptor probe having at least 95% sequence identity to SEQ ID NO: 8. In embodiments, the parC donor-acceptor pair can comprise a donor probe having at least 97% sequence identity to SEQ ID NO: 3 and an acceptor probe having at least 97% sequence identity to SEQ ID NO: 4. In embodiments, the gyrA donor-acceptor pair can comprise a donor probe having at least 97% sequence identity to SEQ ID NO: 7 and an acceptor probe having at least 97% sequence identity to SEQ ID NO: 8. In embodiments, the parC donor-acceptor pair can comprise a donor probe having at least 99% sequence identity to SEQ ID NO: 3 and an acceptor probe having at least 99% sequence identity to SEQ ID NO: 4. In embodiments, the gyrA donor-acceptor pair can comprise a donor probe having at least 99% sequence identity to SEQ ID NO: 7 and an acceptor probe having at least 99% sequence identity to SEQ ID NO: 8. In embodiments, the parC donor-acceptor pair donor probe can comprise SEQ ID NO: 3 and the acceptor probe comprises SEQ ID NO: 4. In embodiments, the gyrA donor-acceptor pair probe can comprise SEQ ID NO: 7 and the acceptor probe comprises SEQ ID NO: 8. In embodiments, the parC donor-acceptor pair donor probe can consist essentially of SEQ ID NO: 3 and the acceptor probe can consist essentially of SEQ ID NO: 4. In embodiments, the gyrA donor-acceptor pair probe can consist essentially of SEQ ID NO: 7 and the acceptor probe can consist essentially of SEQ ID NO: 8. In embodiments, the parC donor-acceptor pair donor probe can consist of SEQ ID NO: 3 and the acceptor probe can consist of SEQ ID NO: 4. In embodiments, the gyrA donor-acceptor pair probe can consist of SEQ ID NO: 7 and the acceptor probe can consist of SEQ ID NO: 8.

Described herein are methods of detecting Mycoplasma genitalium (M. genitalium) in a sample. In embodiments, a method of detecting Mycoplasma genitalium (M. genitalium) in a sample can comprise performing a polymerase chain reaction (PCR) on isolated deoxyribonucleic acid (DNA) from a biological sample; contacting the isolated DNA undergoing PCR with one or more parC donor-acceptor pairs, one or more gyrA donor-acceptor pairs, or both, wherein the one or more parC donor-acceptor pairs comprise a donor probe having at least 90% sequence identity to SEQ ID NO: 3 and an acceptor probe having at least 90% sequence identity to SEQ ID NO: 4, and wherein the one or more gyrA donor-acceptor pairs comprise a donor probe having at least 90% sequence identity to SEQ ID NO: 7 and an acceptor probe having at least 90% sequence identity to SEQ ID NO: 8, or both; and detecting one or more emitted FRET signals resulting from energy transfer within the one or more contacted parC donor-acceptor pairs, one or more contacted gyrA donor-acceptor pairs, or both.

In embodiments of methods described herein, the PCR is real-time PCR, quantitative PCR, digital PCR, or multiple PCR. In embodiments of methods described herein, the PCR is a protocol comprising one or more of the pre-incubation, amplification, melting, or cooling steps of Example 7, individually or in any combination of any thereof. In embodiments of methods described herein, the PCR is a protocol consisting essentially of the pre-incubation, amplification, melting, or cooling steps of Example 7. In embodiments of methods described herein, the PCR is a protocol consisting of the pre-incubation, amplification, melting, or cooling steps of Example 7.

In embodiments of methods described herein, the contacting comprises hybridizing the one or more parC donor-acceptor pairs, one or more gyrA donor-acceptor pairs, or both to a single strand of isolated DNA undergoing PCR during the annealing phase. In embodiments of methods described herein, the biological sample is a urine sample, a cervical swab, a vaginal swab, an endotracheal aspirate, an anorectal specimen, or an oropharyngeal specimen. In embodiments of methods described herein, the biological sample is from a subject having or suspected of having a M. genitalium infection. In embodiments of methods described herein, the one or more emitted FRET signals resulting from energy transfer within the parC donor-acceptor pairs comprises an emission wavelength that is different from the emission wavelength from the one or more gyrA donor-acceptor pairs.

Described herein are kits. In embodiments, a kit can comprise a parC donor-acceptor pair, comprising a donor probe having at least 90% sequence identity to SEQ ID NO: 3 and an acceptor probe having at least 90% sequence identity to SEQ ID NO: 4; a gyrA donor-acceptor pair, comprising a donor probe having at least 90% sequence identity to SEQ ID NO: 7 and an acceptor probe having at least 90% sequence identity to SEQ ID NO: 8; or both; and instructions for use. In embodiments, regarding donor/acceptor FRET pairs, a kit may only contain a parC donor-acceptor pair described herein or may only contain a a gyrA donor-acceptor pair. In embodiments, kits can comprise one or more parC donor-acceptor pairs and one or more gyrA donor-acceptor pairs. In embodiments, a kit can comprise one or more primer pairs configured to amplify a region of parC, one or more primer pairs configured to amplify a region of gyrA, or both. In embodiments, the one or more primer pairs configured to amplify a region of parC can comprise a primer having at least 90% sequence identity to SEQ ID NO: 3 and a primer having at least 90% sequence identity to SEQ ID NO: 4; and the one or more primer pairs configured to amplify a region of gyrA can comprise a primer having at least 90% sequence identity to SEQ ID NO: 3 and a primer having at least 90% sequence identity to SEQ ID NO: 4.

In embodiments of kits described herein, parC donor-acceptor pair can comprise a donor probe having at least 95% sequence identity to SEQ ID NO: 3 and an acceptor probe having at least 95% sequence identity to SEQ ID NO: 4; and wherein the gyrA donor-acceptor pair comprises a donor probe having at least 95% sequence identity to SEQ ID NO: 7 and an acceptor probe having at least 95% sequence identity to SEQ ID NO: 8.

In embodiments of kits described herein, parC donor-acceptor pair can comprise a donor probe having at least 97% sequence identity to SEQ ID NO: 3 and an acceptor probe having at least 97% sequence identity to SEQ ID NO: 4; and wherein the gyrA donor-acceptor pair comprises a donor probe having at least 97% sequence identity to SEQ ID NO: 7 and an acceptor probe having at least 97% sequence identity to SEQ ID NO: 8.

In embodiments of kits described herein, parC donor-acceptor pair can comprise a donor probe having at least 99% sequence identity to SEQ ID NO: 3 and an acceptor probe having at least 99% sequence identity to SEQ ID NO: 4; and wherein the gyrA donor-acceptor pair comprises a donor probe having at least 99% sequence identity to SEQ ID NO: 7 and an acceptor probe having at least 99% sequence identity to SEQ ID NO: 8.

In embodiments of kits described herein, parC donor-acceptor pair can comprise a donor probe having 100% sequence identity to SEQ ID NO: 3 and an acceptor probe having 100% sequence identity to SEQ ID NO: 4; and wherein the gyrA donor-acceptor pair comprises a donor probe having 100% sequence identity to SEQ ID NO: 7 and an acceptor probe having 100% sequence identity to SEQ ID NO: 8.

Also described herein are compositions comprising any one or more components of aspects of the present disclosure, for example, a composition of section (I) (C) below.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A and 1B show quinolone resistance determining region (QRDR) of parC and gyrA.

FIGS. 2A and 2B: MGQR PCR Mutation Detection. Mutant and WT sequences of parC and gyrA were cloned into pUC57 plasmids. A) Twelve parC mutations likely to be associated with fluoroquinolone resistance can be differentiated from WT. B) Ten reported gyrA mutations possibly associated with fluoroquinolone resistance can be differentiated from WT.

FIGS. 3A and 3B: MGQR PCR analytical sensitivity tested by DNA dilution. A) Limit of detection for MGQR-parC PCR is 3.6×10-2 genome copy/μL in PCR mixture, or 1 genome/test. B) LOD for MGQR-gyrA PCR is 0.297 genome/μL in PCR mixture, or 6 genomes/test. Calculation based on the dilution of genome 89607.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genetics, microbiology, biochemistry, molecular biology, cellular biology, tissue culture, therapeutic administrations and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents that otherwise may be present, for example, separated from bacterial stains or species that are not desired, or separating from other constituents that may be present with the micro-organisms in nature.

As used herein, the term “encode” refers to principle that DNA can be transcribed into RNA, which can then be translated into amino acid sequences that can form proteins

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.”

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and includes cells in cell culture, cell supernatants, cell lysates, serum, plasma, biological fluids, and tissue samples. In embodiments, the biological sample comprises urine; a cervical or vaginal swab; or an endotracheal aspirate.

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or more higher.

The terms “administering,” “delivering,” and “introducing,” can be used interchangeably to indicate the introduction of a therapeutic composition or agent (e.g., compositions comprising one or more bacterial species as described herein) into the body of a subject. The therapeutic composition or agent can be administered through any appropriate means that results in the delivery of at least a portion of the composition or agent to a desired location in the subject such that the composition or agent retains its therapeutic capability. Useful methods of delivering the therapeutic include, but are not limited to, intravenous delivery, subcutaneous delivery, intradermal delivery, intracoronary delivery, intracardiac delivery, oral delivery, or any combination thereof.

The term “administered continuously” refers to the continuous delivery of a therapeutic agent, e.g., compound, molecule, peptide, biologic, chemical, etc. over a 24-hour period.

The term “therapeutically effective amount” refers to an amount of therapeutic agent effective to treat at least one symptom of a disease or disorder in a subject. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, CRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23 (4): 581-87 (2013); Xie et al. “Adding amino acids to the genetic repertoire,” Curr. Opin. Chem. Biol. 9 (6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-10 and Altschul et al. (1977) Nucleic Acids Res. 25:3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The terms “co-administration” or “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

Those skilled in the art will appreciate that the term “composition”, as used herein, can be used to refer to a discrete physical entity that comprises one or more specified components. The term “composition” as used herein refers to a product comprising any one or more of the specified ingredients disclosed herein in any one of more of the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier. In general, unless otherwise specified, a composition can be of any suitable form—e.g., gel, liquid, solid, etc.

A composition of the disclosure can be a liquid solution, suspension, emulsion or a powder. Various delivery systems are known and can be used to administer a composition of the disclosure, e.g. encapsulation in liposomes, microparticles, microcapsules, and the like, and then delivered to a patient by means of such as a nebulizer.

Compositions for administration may include sterile aqueous or non-aqueous solvents, such as water, isotonic saline, isotonic glucose solution, buffer solution, or other solvents conveniently used for parenteral administration of therapeutically active agents, stabilizers, buffers, or preservatives, e.g. antioxidants such as methylhydroxybenzoate or similar additives.

A composition of the disclosure may be sterilized by, for example, addition of sterilizing agents to the composition, irradiation of the composition, or heating the composition. Alternatively, the compounds or compositions of the present disclosure may be provided as sterile solid preparations e.g. lyophilized powder, which are readily dissolved in sterile solvent immediately prior to use.

The term “freeze-dried (lyophilized) as used herein refers to a preparation of bacterial cells that have been initially frozen and the water content removed by vacuum.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “preventing” means to stop or hinder a disease, disorder, or symptom of a disease or condition through some action.

The term “reducing” means to diminish in extent, amount, or degree.

The terms “treating” or “treatment” as used herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms or prevents or provides prophylaxis for the disorder or condition.

### Discussion

Described herein are primers, probes, kits, compositions, and methods related to detection of Mycoplasma genitalium (MG) and mutations in quinolone resistance determining region (QRDR) of parC (topoisomerase IV) and gyrA (gyrase). As used herein, parC and gyrA refer to genes of Mycoplasma genitalium.

In certain aspects, described herein is the detection of the presence of the sexually transmitted pathogen Mycoplasma genitalium in clinical specimens (urine, cervical or vaginal swabs, endotracheal aspirates, anorectal and oropharyngeal specimens). In certain aspects, described herein is the detection of the mutations associated fluoroquinolone resistance in Mycoplasma genitalium in clinical specimens (urine, cervical or vaginal swabs, and endotracheal aspirates, anorectal and oropharyngeal specimens).

### I. PRIMERS AND PROBES

Described herein are primers and fluorescence resonance energy transfer (FRET) probes for the detection of gyrase and topoisomerase IV of c. Oligonucleotide primers as described herein can be conjugated to a FRET probe, with one primer of a primer pair comprising a donor probe and another primer of the primer pair comprising an acceptor or donor, respectively. As used herein, a primer is an oligonucleotide that can be utilized for polymerase chain reaction and hybridization to a single strand of DNA that lacks a donor or acceptor molecule described herein. As used herein, a FRET probe or donor probe or acceptor probe (or donor/acceptor FRET pair or FRET primer pair) refers to a primer that is conjugated to a donor probe at the 3′ end or an acceptor probe at the 5′ end.

### A. Probes

Described herein are oligonucleotide primers conjugated to a donor or acceptor molecule (for example, a small organic dye, fluorescent proteins (FP), or quantum dots (QD)) that can be utilized as FRET probe pair that can be used for detection of Mycoplasma genitalium (MG). in particular, mutations associated with quinolone resistance in the parC region of M. genitalium topoisomerase IV (for example, gyrA region of M. genitalium gyrase.

### a) Sequences

FRET Examples of embodiments of primers according to the present disclosure are listed below:

TABLE 1
Informal Sequence Listing

| | | 5′ | 3′ | SEQ ID |
|---|---|---|---|---|
| Primer/Probe | Sequence (5′→3′) | Label | Label | NO: |
| MGPARC-A | TGGGCTTAAAACCCACCACT | | | 1 |
| (forward) | | | | |
| MGPARC-B | CGGGTTTCTGTGTAACGCAT | | | 2 |
| (reverse) | | | | |
| MGQR-hyb-P1 | CCCCATGGTGATAGTTCCATTTA | | Fluor- | 3 |
| | TGATGCAAT* | | escein | |
| MGQR-hyb-P2 | *ATCAGAATGTCCCAAAGCTGAA | LC Red | Phos- | 4 |
| | AGAACAACTG | 610 | phate | |
| MG-GYRA-A2 | TCGTCGTGTTCTTTATGGTGC | | | 5 |
| (forward) | | | | |
| MG-GYRA-B | ATAACGYYGTGCAGCAGGTC | | | 6 |
| (reverse) | | | | |
| MGQR-GYRA-P1- | TTCTTGACATGGTGTCATATATTG | | Fluor- | 7 |
| ssr | CCATATCACCA* | | escein | |
| MGQR-GYRA-P1- | *AGGGTGGAATTTACTCATTACAT | LC Red | Phos- | 8 |
| a | CACCAACAATCC | 610 | phate | |

*indicates presence of donor or acceptor molecule (i.e., a dye or fluorophore)

In embodiments, primers according to the present disclosure can have about 75%, 80%, 85%, 90%, 91%, 92%, 93%, 94%, 95%, 96%, 97%, 98%, 99% or 100% sequence identify with any one or more of SEQ ID NOs: 1-8 disclosed above. Any one or more primers according to the present disclosure can also be reverse of the primers above, the complement of the primers above, or the reverse-complement of the primers above. In embodiments, primers according to the present disclosure can have about 75%, 80%, 85%, 90%, 91%, 92%, 93%, 94%, 95%, 96%, 97%, 98%, 99% or 100% sequence identify with the reserve of the primers above, the complement of the primers above, or the reverse-complement any one or more of SEQ ID NOs: 1-8 disclosed above in Table 1.

parC FRET donor/acceptor pairs described herein are designed in particular to detect one or more of the following 12 mutations compared to wild-type (for example, NCBI Gene ID: 99647045): G241T, G244A, A247C, G248A, G248T, T249A, T250C, G259A, G259C, G259T, A260G, or A260T, individually or in any combination of any thereof. In embodiments parC FRET donor/acceptor pairs described herein can detect all 12 mutations.

gyrA FRET donor/acceptor pairs described herein are designed in particular to detect one or more of the following 12 mutations compared to wild-type: G277T, A283G, G285A, G285C, G285T, G286A, G295A, G295T, A296C, or A296G, individually or in any combination of any thereof. In embodiments gyrA FRET donor/acceptor pairs described herein can detect all 12 mutations.

In embodiments, a parC FRET donor/acceptor pair can comprise a pair of sequences having at least 75%, 80%, 85%, 90%, 91%, 92%, 93%, 94%, 95%, 96%, 97%, 98%, 99% or 100% sequence identity to each of SEQ ID NOs: 3 and 4 (or the reverse, complement, or reverse complement thereof).

In embodiments, a parC FRET donor/acceptor pair can consist essentially of (i.e., hybridize to a strand of a region of parC and detect one or mutations discussed above) two sequences having at least 75%, 80%, 85%, 90%, 91%, 92%, 93%, 94%, 95%, 96%, 97%, 98%, 99% or 100% sequence identity to each of SEQ ID NOs: 3 and 4 (or the reverse, complement, or reverse complement thereof).

In embodiments, a parC FRET donor/acceptor pair can consist of (i.e., hybridize to a strand of a region of parC and detect one or mutations discussed above) two sequences having at least 75%, 80%, 85%, 90%, 91%, 92%, 93%, 94%, 95%, 96%, 97%, 98%, 99% or 100% sequence identity to each of SEQ ID NOs: 3 and 4 (or the reverse, complement, or reverse complement thereof).

In embodiments, a gyrA FRET donor/acceptor pair can comprise two sequences having at least 75%, 80%, 85%, 90%, 91%, 92%, 93%, 94%, 95%, 96%, 97%, 98%, 99% or 100% sequence identity to each of SEQ ID NOs: 7 and 8 (or the reverse, complement, or reverse complement thereof).

In embodiments, a gyrA FRET donor/acceptor pair can consist essentially of (i.e., hybridize to a strand of a region of gyrA and detect one or mutations discussed above) two sequences having at least 75%, 80%, 85%, 90%, 91%, 92%, 93%, 94%, 95%, 96%, 97%, 98%, 99% or 100% sequence identity to each of SEQ ID NOs: 7 and 8 (or the reverse, complement, or reverse complement thereof).

In embodiments, a gyrA FRET donor/acceptor pair can consist of (i.e., hybridize to a strand of a region of gyrA and detect one or mutations discussed above) two sequences having at least 75%, 80%, 85%, 90%, 91%, 92%, 93%, 94%, 95%, 96%, 97%, 98%, 99% or 100% sequence identity to each of SEQ ID NOs: 7 and 8 (or the reverse, complement, or reverse complement thereof).

### b) Donor and Acceptor Molecules

In an embodiment, primers according to the present disclosure can have one primer with a 3′ donor probe at the end of the sequence, and a second primer can have a 5′ acceptor probe at the beginning of the sequence (the primer plus donor or acceptor molecule together also referred to herein as a FRET probe). The second primer with 5′ acceptor can also have a phosphate or phosphate cap on the 3′ terminal end. Primers according to the table above can be conjugated to a 3′ probe or a 5′ probe. As used herein, donor probe and acceptor probe, or donor molecule and acceptor molecule, refer to, for example, fluorophores or fluorescent proteins that are chemically conjugated to the polynucleotide sequences described herein. In embodiments, a FRET probe conjugated to the 5′ or 3′ end of an oligonucleotide described herein to create a FRET donor or acceptor probe can be a small organic dye, fluorescent protein (FP), and/or quantum dots (QD).

Examples of FRET donor/acceptor pairs include eCFP and eYFP or mClover3 and mRuby3. Additional examples of FRET fluorescent donor and acceptor probe pairs can be found, for example, in Bajar B T, Wang E S, Zhang S, Lin M Z, Chu J. A Guide to Fluorescent Protein FRET Pairs. Sensors (Basel). 2016 Sep. 14; 16 (9): 1488. doi: 10.3390/s16091488. PMID: 27649177; PMCID: PMC5038762, the contents of which are incorporated by reference as if fully set forth herein regarding donor and acceptor FRET probe pairs. In embodiments, a probe or probe pair can be a LightCycler® probe or probe pair from Millipore Sigma.

Other non-limiting examples of donor probes include LC610, LC640, LC670, LC705, LC Fluro, Cy® 5, Cy® 5.5 3′ FAM. Other examples of acceptor probes include LC® Cyan 500, LC® Fluo, Texas Red, LC® Red 610, LC® Red 640, LC® Red 670, ROX, and Fluorescein. A phosphate can be added to the 3′ end of the primer with the 5′ acceptor probe.

### B. Primers

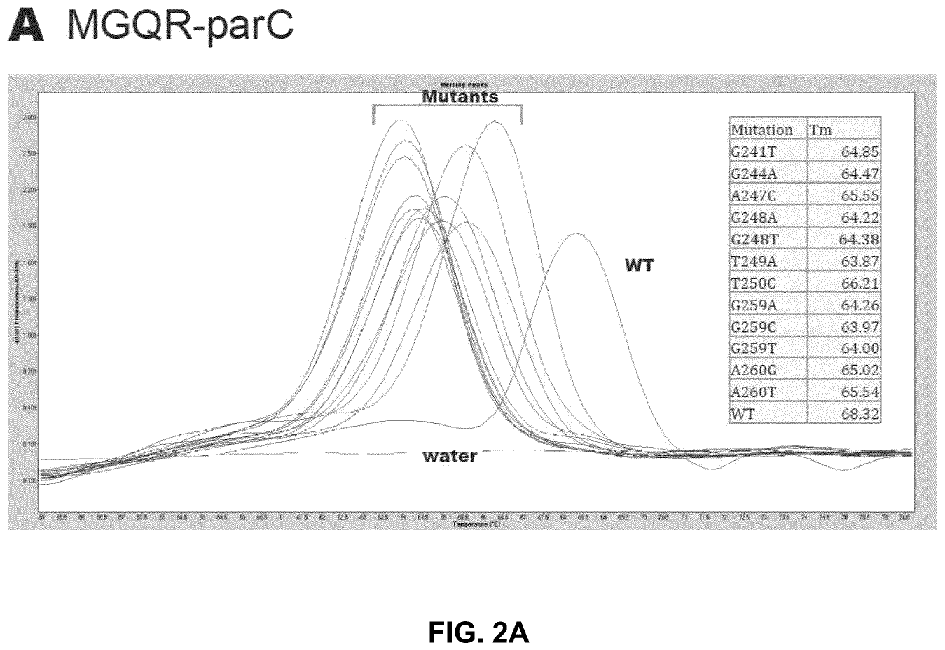

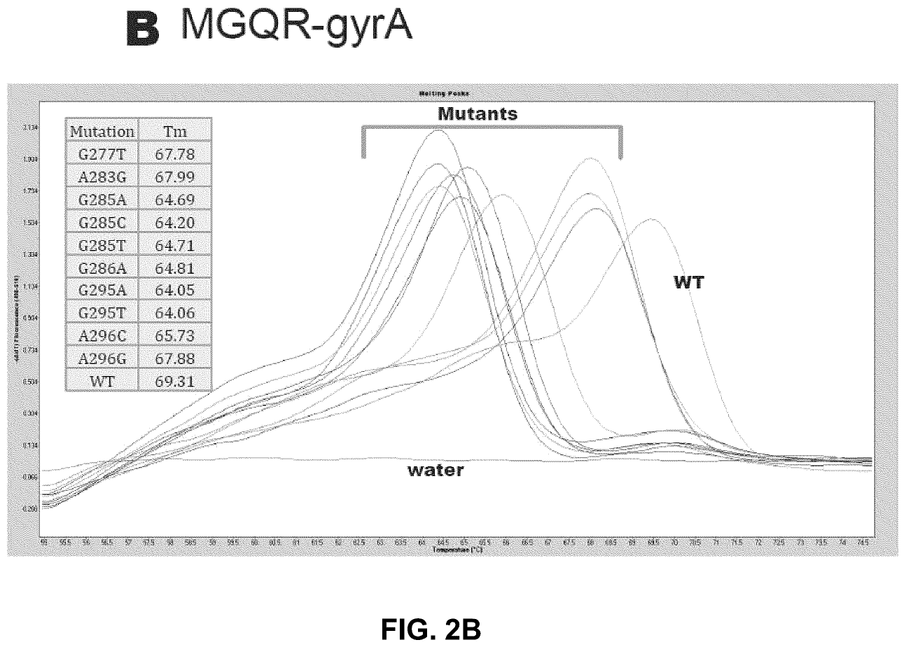

Also described herein are primers for the amplification of a target region of parC, gyrA, or both. A target region of parC, gyrA, or both that can be amplified can be found, for example, in FIGS. 2A and 2B of U.S. Provisional Application No. 63/713,308.

Such primers and primer pairs for the amplification of a target region of parC, gyrA, or both described herein can comprise a pair of oligonucleotides (a forward primer and reverse primer) that can be utilized with any PCR technique known in the art. Primers as described in the present section lack conjugation to one or more of a donor or acceptor FRET fluorescent protein or fluorophore discussed in section (A) above).

In embodiments, a primer pair for amplification of a target region of parC can comprise two sequences having at least 75%, 80%, 85%, 90%, 91%, 92%, 93%, 94%, 95%, 96%, 97%, 98%, 99% or 100% sequence identity to two of SEQ ID NOs: 1 to 2 (or the reverse, complement, or reverse complement thereof). It is understood that such primer pairs are not FRET primer/probe pairs.

In embodiments, a primer pair for amplification of a target region of parC can consist essentially of (i.e., deviant in sequence by a small degree but not so large a degree to present specific amplification of a target region of parC) two sequences having at least 75%, 80%, 85%, 90%, 91%, 92%, 93%, 94%, 95%, 96%, 97%, 98%, 99% or 100% sequence identity to two of SEQ ID NOs: 1 to 2 (or the reverse, complement, or reverse complement thereof). It is understood that such primer pairs are not FRET primer/probe pairs.

In embodiments, a primer pair for amplification of a target region of parC can consist of two sequences having at least 75%, 80%, 85%, 90%, 91%, 92%, 93%, 94%, 95%, 96%, 97%, 98%, 99% or 100% sequence identity to two of SEQ ID NOs: 1 to 2 (or the reverse, complement, or reverse complement thereof). It is understood that such primer pairs are not FRET primer/probe pairs.

In embodiments, a primer pair for amplification of a target region of gyrA can comprise two sequences having at least 75%, 80%, 85%, 90%, 91%, 92%, 93%, 94%, 95%, 96%, 97%, 98%, 99% or 100% sequence identity to two of SEQ ID NOs: 5 and 6 (or the reverse, complement, or reverse complement thereof). It is understood that such primer pairs are not FRET primer/probe pairs.

In embodiments, a primer pair for amplification of a target region of gyrA can consist essentially of (i.e., deviant in sequence by a small degree but not so large a degree to present specific amplification of a target region of gyrA) two sequences having at least 75%, 80%, 85%, 90%, 91%, 92%, 93%, 94%, 95%, 96%, 97%, 98%, 99% or 100% sequence identity to two of SEQ ID NOs: 5 and 6 (or the reverse, complement, or reverse complement thereof). It is understood that such primer pairs are not FRET primer/probe pairs.

In embodiments, a primer pair for amplification of a target region of gyrA can consist of two sequences having at least 75%, 80%, 85%, 90%, 91%, 92%, 93%, 94%, 95%, 96%, 97%, 98%, 99% or 100% sequence identity to two of SEQ ID NOs: 5 and 6 (or the reverse, complement, or reverse complement thereof).

The skilled artisan would understand that amplification primers described herein are not necessarily limited to those described herein. A number of tools widely and freely available to design primers to amplify a target region are available that can design suitable target-region amplification primers, for example and without intending to be limiting: NCBI Primer-BLAST (https://www.ncbi.nlm.nih.gov/tools/primer-blast/), Oligo Design Tools from Thermo Fisher® (https://www.thermofisher.com/us/en/home/life-science/oligonucleotides-primers-probes-genes/custom-dna-oligos/oligo-design- tools.html), Primer design tools for PCR & qPCR from IDT® (https://www.idtdna.com/page/support-and-education/decoded-plus/design-efficient-pcr-and-qpcr-primers-and-probes-using-online-tools/), Primer Design Tool from Vector Builder (https://en.vectorbuilder.com/tool/primer-design.html), and the like.

### C. Compositions

Compositions as described herein can comprise a parC FRET donor/acceptor pair, gyrA FRET donor/acceptor pair, or both. Compositions as described herein can further comprise a primer pair for amplification of a target region of parC, gyrA, or both. A target region of parC, gyrA, or both that can be amplified can be found, for example, in FIGS. 2A and 2B of U.S. Provisional Application No. 63/713,308.

Compositions as described herein can further comprise any one or more of buffers, PCR-grade water, master mixes, DNA polymerases, deoxynucleotide triphosphates (dNTPs), Mg²⁺, and any one or more other additional reagents that are typically employed in PCR reactions, individual or in any combination of any thereof.

In other embodiments, compositions further comprise a FRET donor/acceptor probe pair in addition to a “master mix,” for example, a LightCycler® 480 Probes Master 2× master mix. In other embodiments, compositions further comprise a FRET donor/acceptor probe pair in addition to an amplification primer pair and a “master mix,” for example, a LightCycler® 480 Probes Master 2× master mix. Additional details of such compositions can be found, for example, in Examples 6 and 7.

### II. METHODS

Described herein are real-time polymerase chain reaction (PCR) methods utilizing primers and donor/acceptor FRET pairs to monitor Mycoplasma genitalium proliferation, amount, and mutations associated with quinolone-resistance. Methods as described herein can be utilized, for example, in clinical and research settings with biological samples that include, for example, urine samples, cervical swabs, vaginal swabs, endotracheal aspirates, anorectal specimens, or oropharyngeal specimens. In embodiments, the biological sample can be from a subject having or suspected of having a M. genitalium infection.

Generally, step 1 is a PCR reaction that results in amplification of a fragment with an incorporated donor fluorophore attached to the forward primer. Step 2: After amplification, the acceptor fluorophore-labeled probe is added to the reaction and during initial denaturation hybridizes to the amplicon. Step 3: The two fluorophores are now in close proximity of each other and energy from the excited donor is transferred to the acceptor generating the FRET signal. Step 4. The increase of temperature during melt curve analysis leads, at a specific temperature, to the dissociation of the probe from the amplicon. When the probe is dissociated transfer of energy is lost and therefore no FRET signal can be observed.

PCR can be performed with denaturation, annealing, and extension, with repeats of cycles of these three steps.

During denaturation, the temperature increases to around 95-98° C. for 15-30 seconds. The high temperature causes the double-stranded DNA template to denature, resulting in two single-stranded DNA templates.

Annealing involves lowering the temperature to around 50-56° C. for specific amplification and 36° C. for a specific amplification. At this temperature, the primers bind to the single-stranded DNA templates to provide a starting point for the polymerase enzyme.

For extension the temperature is raised to around 72-75° C. for 15-30 seconds during this step. The polymerase enzyme extends the primers by adding nucleotides to the 3′ end of the DNA template, creating a new double-stranded DNA molecule.

This process can then be repeated. Other times and temperatures can be used, for example, 0 s at 85 degrees C. for denaturation, 7 s for annealing, and 2 s for elongation.

Additional information on FRET methodology can be found, for example, in Ahmad Al, Ghasemi J B. New FRET primers for quantitative real-time PCR. Anal Bioanal Chem. 2007 April; 387 (8): 2737-43. doi: 10.1007/s00216-007-1123-4. Epub 2007 Feb. 17. PMID: 17308892, the contents of which are incorporated by reference as if fully set forth herein. An embodiment of a FRET PCR is shown on page 20 of U.S. Provisional Application No. 63/713,308.

Aspect of use of LightCycler® FRET can be found, for example, in Hardick J, Yang S, Lin S, Duncan D, Gaydos C. Use of the Roche LightCycler® instrument in a real-time PCR for Trichomonas vaginalis in urine samples from females and males. J Clin Microbiol. 2003 December; 41(12): 5619-22. doi: 10.1128/JCM.41.12.5619-5622.2003. PMID: 14662951; PMCID: PMC309023.

In additional methods, the biological sample can be provided. In additional embodiments, methods can comprise isolating DNA from a biological sample prior to performing PCR. DNA isolation from samples described herein can be performed according to known methods in the art and with kits known in the art, for example, urine and/or blood or tissue DNA collection kits that are commercially available.

In further embodiments, a target region of parC, gyrA, or both can be amplified, and the FRET donor/acceptor pair can be added to the reaction mixture after a certain number of cycles (25-35 cycles, for example), at which points the FRET donor/acceptor pair probes can hybridize to a single strand of amplified target DNA during the annealing phase.

Upon hybridization of the FRET donor/acceptor pair to the single stranded DNA of a sample, energy can be transferred and a signal detected that is representative of the energy transfer or wavelength shift of emission caused by pairing of the donor/acceptor. Presence of a signal induced by the energy transfer indicates the presence of Mycoplasma genitalium in a subject. In particular, presence of a signal can indicate the presence of any one or more mutations (one or more of 12 mutations in parC compared to wild-type and/or one or more of 10 mutations in gyrA compared to wild-type, or the presence of all mutations), which can indicate the presence of a macrolide and/or fluoroquinolone resistant strain of Mycoplasma genitalium in the sample.

Additional details of and embodiments of methods according to the present disclosure can be found, for example, in Examples 4-7 below.

### III. KITS AND PACKAGING

Described herein are kits comprising primers for FRET PCR, in addition to containers for storage and/or use, and instructions for use. Kits as described herein can comprise any one or more FRET donor/acceptor pairs for parC and any one or more FRET donor/acceptor pairs for gyrA, or both. Kits as described herein can further comprise any one or more primer pairs for the amplification of a target region of parC, gyrA, or both.

Kits as described herein can further comprise any one or more reagents for the isolation of DNA from a biological sample comprising samples, for example, a lysis enzyme and associated buffers known in the art.

Kits as described herein can further comprise any one or more traditional reagents for PCR, for example, DNA polymerase[s], buffers, water, Mg²⁺, dNTPs, and the like. Other components of kits can be seen, for example, in Examples 5 and 6 below.

The kit can be a package which houses a container which contains compositions of the disclosure or formulations of the disclosure and also houses instructions for using such. The disclosure further relates to a commercial package comprising compounds of the disclosure or formulations of the disclosure together with instructions for simultaneous, separate or sequential use.

The disclosure also relates to articles of manufacture and kits containing materials useful for diagnosing or treating a disease disclosed herein. An article of manufacture may comprise a container with a label. Examples of suitable containers include bottles, vials, and test tubes, or a delivery device such as a nebulizer, which may be formed from a variety of materials including glass and plastic. A container holds compounds of the disclosure or formulations of the disclosure which are effective for treating a disease disclosed herein. The label on the container indicates that the compounds of the disclosure or formulations of the disclosure are used for treating a disease disclosed herein and may also indicate directions for use. In aspects of the disclosure, a medicament or formulation in a container may comprise any of the medicaments or formulations disclosed herein.

The disclosure also contemplates kits comprising one or more of oligonucleotides or any other reagents described herein of the disclosure. In aspects of the disclosure, a kit of the disclosure comprises a container described herein. In particular aspects, a kit of the disclosure comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disease disclosed herein).

The compositions (i.e., those comprising, consisting essentially of, or consisting of primers and probes and pairs described herein) can be utilized in the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the methods.

In some instances, one of the containers may comprise a composition as described in this disclosure that is, or can be, detectably labeled (for example with a donor or acceptor FRET probe). The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises separate containers containing compositions described herein and a detectable label.

### IV. EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1: Additional Details

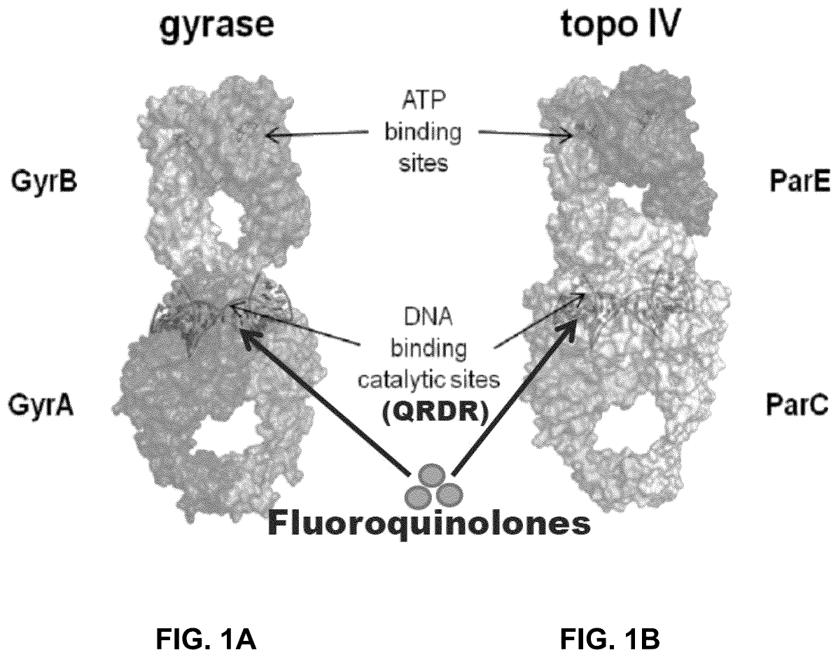

FIGS. 1A and 1B show quinolone resistance determining region (QRDR) of parC and gyrA.

FIGS. 2A and 2B: MGQR PCR Mutation Detection. Mutant and WT sequences of parC and gyrA were cloned into pUC57 plasmids. A) Twelve parC mutations likely to be associated with fluoroquinolone resistance can be differentiated from WT. B) Ten reported gyrA mutations possibly associated with fluoroquinolone resistance can be differentiated from WT.

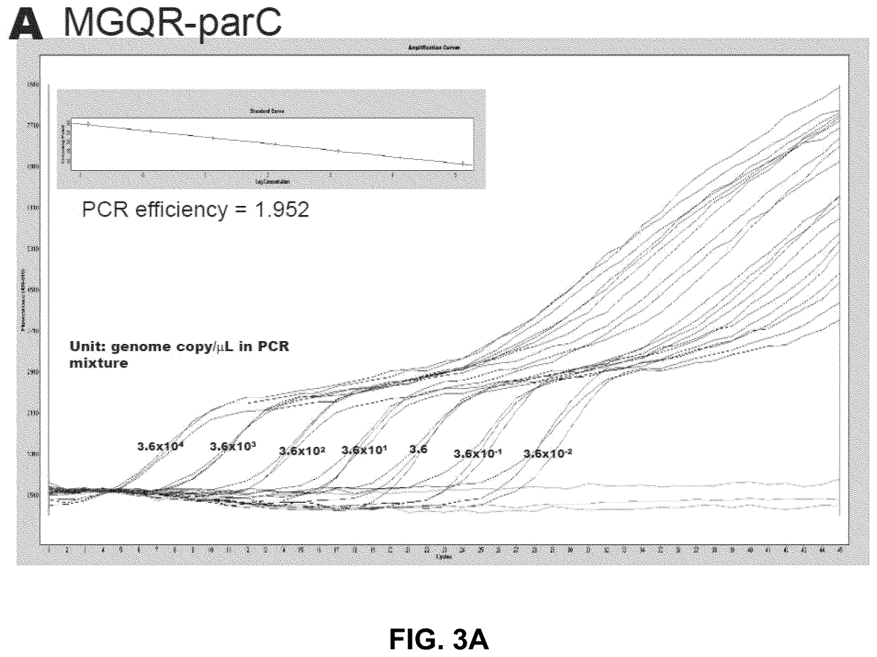

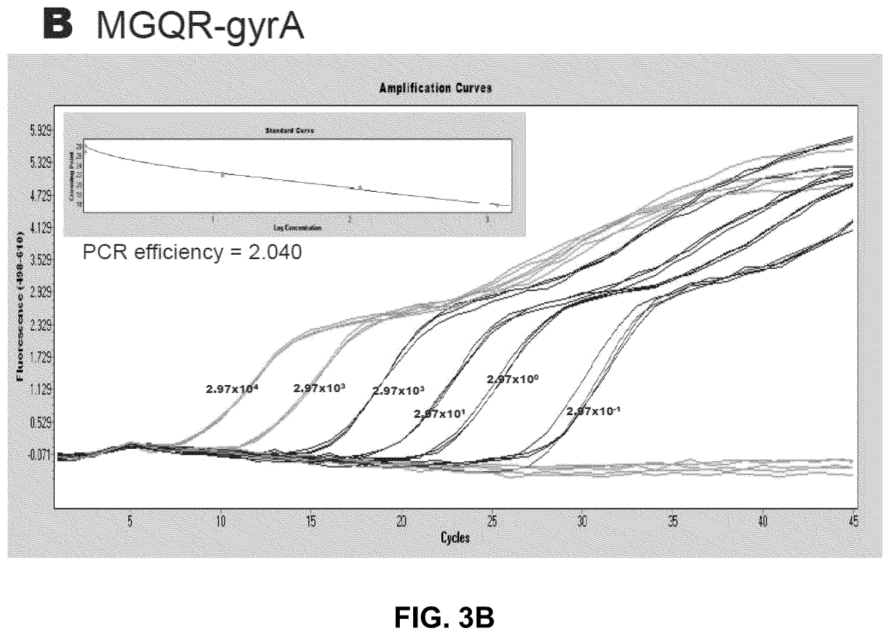

FIGS. 3A and 3B: MGQR PCR analytical sensitivity tested by DNA dilution. A) Limit of detection for MGQR-parC PCR is 3.6×10-2 genome copy/μL in PCR mixture, or 1 genome/test. B) LOD for MGQR-gyrA PCR is 0.297 genome/μL in PCR mixture, or 6 genomes/test. Calculation based on the dilution of genome 89607.

### Example 2: Additional Details

FIGS. 2A and 2B of U.S. Provisional Application No. 63/713,308 show design of embodiments of probes for two real-time PCRs using Light Cycler hybridization FRET. FIGS. 2A and 2B are incorporated by reference (as part of U.S. Provisional Application No. 63/713,308 as filed) as previously noted in the CROSS-REFERENCE TO RELATED APPLICATION[S] section on the first page.

### Example 3: Additional Details

Additional details of the present disclosure can be found, for example, in Table 1 of Exhibit A of U.S. Provisional Application No. 63/713,308, which is incorporated by reference (as part of U.S. Provisional Application No. 63/713,308 as filed) as previously noted in the CROSS-REFERENCE TO RELATED APPLICATION[S] section on the first page.

20 tests on both WT and mutant strains at different dilutions on different test days.
MGQR-parC PCR results are reproducible at dilutions 10-3 to 10-6 (strain G37, WT) and 10-3 to 10-7 (strain 75956, mutant); and
MGQR-gyrA PCR results are reproducible at dilutions 10-3 to 10-6 for WT strain G37 and mutant strain 89607.

Data is shown in Table 1 of Exhibit A of U.S. Provisional Application No. 63/713,308.

The following was noted:

MGQR PCR assay has a limit of detection of 1 and 6 genomes per test for parC and gyrA, respectively.

There was no cross reaction with other organisms.

Aspects of the present disclosure detect all currently reported QRDR mutations related to fluoroquinolone treatment failure but avoids the most frequent synonymous mutations (C234T, for example) in parC.

Aspects of the present disclosure were validated for variety of sample types (urine, cervical, vaginal, and endotracheal aspirates).

Aspects of the present disclosure exhibit a sensitivity of 98.5% and specificity of 95.9% for MG detection in clinical specimens compared to reference method MGMR PCR and 100% sensitivity and specificity for identifying QRDR mutations compared to Sanger sequencing.

As demonstrated, MGQR PCR is sensitive and specific for simultaneously detecting MG and mutations associated with fluoroquinolone resistance in parC and gyrA genes. It is suitable for research and clinical test purposes to detect macrolide resistance, for example.

### Example 4: Additional Details

Additional details of the present disclosure can be found, for example, in FIGS. 5A-5C of U.S. Provisional Application No. 63/713,308, which is incorporated by reference as previously noted in the CROSS-REFERENCE TO RELATED APPLICATION[S] section on the first page.

### Example 5: Additional Details of parC PCR Reagent Mixtures

An embodiment of reagent mixtures for parC PCR assays (MGQR-parC) can be found below:

TABLE 2
An embodiment of reagent mixtures for parC PCR assays.
The LC480 Probes Master 2x can be a master mix from
Roche, the LightCycler ® 480 Probes Master,
for example. Template = DNA from a biological
sample, in particular isolated DNA from a sample.

| PCR Master Mix: | | Vol (μL) |
|---|---|---|
| PCR grade water | | 2.20 |
| MGparCA (10 uM) | SEQ ID | 0.40 |
| | NO: 1 | |
| MGparCB (10 uM) | SEQ ID | 1.20 |
| | NO: 2 | |
| MGQR-hyb-P1 (10 uM) | SEQ ID | 0.60 |
| | NO: 3 | |
| MGQR-hyb-P2 (10 uM, LR610) | SEQ ID | 0.60 |
| | NO: 4 | |
| LC480 Probes Master 2X | | 10.00 |
| Total Volume | | 15.00 |
| Template: | | 5.00 |

### Example 6: Additional Details of gyrA PCR Reagent Mixtures

An embodiment of reagent mixtures for gyrA PCR assays (MGQR-gyrA-sa) can be found below:

TABLE 3
An embodiment of reagent mixtures for gyrA PCR assays.
The LC480 Probes Master 2x can be a master mix from
Roche, the LightCycler ® 480 Probes Master,
for example. Template = DNA from a biological
sample, in particular isolated DNA from a sample.

| PCR Master Mix: | | Vol (μL) |
|---|---|---|
| PCR grade water | | 2.20 |
| MGgyrA-A2 (10 uM) | SEQ ID | 1.00 |
| | NO: 5 | |
| MGgyrA-B (10 uM) | SEQ ID | 0.40 |
| | NO: 6 | |
| MGGyrA-P1-ssr (10 uM) | SEQ ID | 0.70 |
| | NO: 7 | |
| MGgyrA-P2-ach (10 uM, LR610) | SEQ ID | 0.70 |
| | NO: 8 | |
| LC480 Probes Master 2X | | 10.00 |
| Total Volume | | 15 |
| Template: | | 5.00 |

### Example 7: Additional Details of gyrA PCR Protocols

An embodiment of a PCR protocol according to the present disclosure is as follows, for example, to be employed on a LightCycler® machine:

### Pre-Incuation:

40° C., 10 min; 95° C., 10 min; 4.4° C./s.

### Amplification:

I: 95° C., 10 s, 4.4° C./s; 60° C., 10 s, 1.1° C./s; 72° C., 15 s, 4.4° C./s; 10 cycles.
II: 95° C., 10 s, 4.4° C./s; 60° C., 10 s, 2.2° C./s; 72° C., 15 s, 4.4° C./s; step from 60° C. to 55° C., 1° C./step; 45 cycles.

### Melting:

95° C., 15 s, 4.4° C./s; 55° C., 15 s, 2.2° C./s; to 77° C.; 0.11° C./s;
Acquisition 5 per° C.

### Cooling:

40° C., 30 s, 2.2° C./s.

### REFERENCES RELATED TO THE PRESENT DISCLOSURE

1. Lau A, Bradshaw C S, Lewis D, et al. The efficacy of azithromycin for the treatment of genital Mycoplasma genitalium: A systematic review and meta-analysis. Clin Infect Dis. 2015; 61:1389-99.
2. Workowski K A, Bachman L H, Chan P A, et al. Sexually Transmitted Infections Treatment Guidelines, 2021.MMWR; RecommRep2021; 70 (No.RR-4:1-87.
3. Xiao L, Waites K B, Wang H, Van Der Pol B, Ratliff A E, Geisler W M. Evaluation of a real-time PCR Assay for detection of Mycoplasma genitalium and macrolide resistance-mediating mutations from clinical specimens. Diagn Microbiol Infect Dis. 2018; 91:123-125.
4. Waites K, Crabb D, Ratliff A, Geisler W M, Atkinson T P, Xiao L. Latest advanced in laboratory deteciton of Mycoplasma genitalium. J Clin Microbiol 2023; 23:61 (3):e0079021
5. Murray G L, Plummer K, Bodiyabadu L A, et al. gyrA mutations in Mycoplasma genitalium and their contribution to moxifloxacin failure: time for the next generation of resistance-guided therapy. Clin Infect Dis 2023; 76:2187-2195.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1. A donor-acceptor fluorescence resonance energy transfer (FRET) probe pair, comprising:
a parC donor-acceptor pair, comprising a donor probe having at least 90% sequence identity to SEQ ID NO: 3 and an acceptor probe having at least 90% sequence identity to SEQ ID NO: 4; or
a gyrA donor-acceptor pair, comprising a donor probe having at least 90% sequence identity to SEQ ID NO: 7 and an acceptor probe having at least 90% sequence identity to SEQ ID NO: 8.

2. The donor-acceptor FRET probe pair of claim 1,
wherein the parC donor-acceptor pair comprises a donor probe having at least 95% sequence identity to SEQ ID NO: 3 and an acceptor probe having at least 95% sequence identity to SEQ ID NO: 4; and
wherein the gyrA donor-acceptor pair comprises a donor probe having at least 95% sequence identity to SEQ ID NO: 7 and an acceptor probe having at least 95% sequence identity to SEQ ID NO: 8.

3. The donor-acceptor FRET probe pair of claim 1,
wherein the parC donor-acceptor pair comprises a donor probe having at least 97% sequence identity to SEQ ID NO: 3 and an acceptor probe having at least 97% sequence identity to SEQ ID NO: 4; and
wherein the gyrA donor-acceptor pair comprises a donor probe having at least 97% sequence identity to SEQ ID NO: 7 and an acceptor probe having at least 97% sequence identity to SEQ ID NO: 8.

4. The donor-acceptor FRET probe pair of claim 1,
wherein the parC donor-acceptor pair comprises a donor probe having at least 99% sequence identity to SEQ ID NO: 3 and an acceptor probe having at least 99% sequence identity to SEQ ID NO: 4; and
wherein the gyrA donor-acceptor pair comprises a donor probe having at least 99% sequence identity to SEQ ID NO: 7 and an acceptor probe having at least 99% sequence identity to SEQ ID NO: 8.

5. The donor-acceptor FRET probe pair of claim 1,
wherein the parC donor-acceptor pair donor probe comprises SEQ ID NO: 3 and the acceptor probe comprises SEQ ID NO: 4; and
wherein the gyrA donor-acceptor pair probe comprises SEQ ID NO: 7 and the acceptor probe comprises SEQ ID NO: 8.

6. The donor-acceptor FRET probe pair of claim 1,
wherein the parC donor-acceptor pair donor probe consists essentially of SEQ ID NO: 3 and the acceptor probe consists essentially of SEQ ID NO: 4; and
wherein the gyrA donor-acceptor pair probe consists essentially of SEQ ID NO: 7 and the acceptor probe consists essentially of SEQ ID NO: 8.

7. The donor-acceptor FRET probe pair of claim 1,
wherein the parC donor-acceptor pair donor probe consists of SEQ ID NO: 3 and the acceptor probe consists of SEQ ID NO: 4; and
wherein the gyrA donor-acceptor pair probe consists of SEQ ID NO: 7 and the acceptor probe consists of SEQ ID NO: 8.

8. A method of detecting Mycoplasma genitalium (M. genitalium) in a sample, comprising:
performing a polymerase chain reaction (PCR) on isolated deoxyribonucleic acid (DNA) from a biological sample;
contacting the isolated DNA undergoing PCR with one or more parC donor-acceptor pairs, one or more gyrA donor-acceptor pairs, or both,
wherein the one or more parC donor-acceptor pairs comprise a donor probe having at least 90% sequence identity to SEQ ID NO: 3 and an acceptor probe having at least 90% sequence identity to SEQ ID NO: 4, and wherein the one or more gyrA donor-acceptor pairs comprise a donor probe having at least 90% sequence identity to SEQ ID NO: 7 and an acceptor probe having at least 90% sequence identity to SEQ ID NO: 8, or both; and
detecting one or more emitted FRET signals resulting from energy transfer within the one or more contacted parC donor-acceptor pairs, one or more contacted gyrA donor-acceptor pairs, or both.

9. The method of claim 8, wherein the PCR is real-time PCR, quantitative PCR, digital PCR, or multiple PCR.

10. The method of claim 8, wherein the PCR is a protocol comprising one or more of the pre-incubation, amplification, melting, or cooling steps of Example 7, individually or in any combination of any thereof.

11. The method of claim 8, wherein the contacting comprises hybridizing the one or more parC donor-acceptor pairs, one or more gyrA donor-acceptor pairs, or both to a single strand of isolated DNA undergoing PCR during the annealing phase.

12. The method of claim 8, wherein the biological sample is a urine sample, a cervical swab, a vaginal swab, an endotracheal aspirate, an anorectal specimen, or an oropharyngeal specimen.

13. The method of claim 8, wherein the biological sample is from a subject having or suspected of having a M. genitalium infection.

14. The method of claim 8, wherein the one or more emitted FRET signals resulting from energy transfer within the parC donor-acceptor pairs comprises an emission wavelength that is different from the emission wavelength from the one or more gyrA donor-acceptor pairs.

15. A kit, comprising:
a parC donor-acceptor pair, comprising a donor probe having at least 90% sequence identity to SEQ ID NO: 3 and an acceptor probe having at least 90% sequence identity to SEQ ID NO: 4;
a gyrA donor-acceptor pair, comprising a donor probe having at least 90% sequence identity to SEQ ID NO: 7 and an acceptor probe having at least 90% sequence identity to SEQ ID NO: 8; or both; and
instructions for use.

16. The kit of claim 15, comprising one or more parC donor-acceptor pairs and one or more gyrA donor-acceptor pairs.

17. The kit of claim 15, further comprising one or more primer pairs configured to amplify a region of parC, one or more primer pairs configured to amplify a region of gyrA, or both.

18. The kit of claim 15, wherein
the one or more primer pairs configured to amplify a region of parC comprise a primer having at least 90% sequence identity to SEQ ID NO: 3 and a primer having at least 90% sequence identity to SEQ ID NO: 4; and
the one or more primer pairs configured to amplify a region of gyrA comprise a primer having at least 90% sequence identity to SEQ ID NO: 3 and a primer having at least 90% sequence identity to SEQ ID NO: 4.

19. The kit of claim 15,
wherein the parC donor-acceptor pair comprises a donor probe having at least 95% sequence identity to SEQ ID NO: 3 and an acceptor probe having at least 95% sequence identity to SEQ ID NO: 4; and
wherein the gyrA donor-acceptor pair comprises a donor probe having at least 95% sequence identity to SEQ ID NO: 7 and an acceptor probe having at least 95% sequence identity to SEQ ID NO: 8.

20. The kit of claim 15,
wherein the parC donor-acceptor pair comprises a donor probe having at least 97% sequence identity to SEQ ID NO: 3 and an acceptor probe having at least 97% sequence identity to SEQ ID NO: 4; and
wherein the gyrA donor-acceptor pair comprises a donor probe having at least 97% sequence identity to SEQ ID NO: 7 and an acceptor probe having at least 97% sequence identity to SEQ ID NO: 8.


SEQUENCE LISTING (8 sequences)

SEQ ID NO: 1 - DNA, 20 bp, synthetic construct
  source 1..20: mol_type = other DNA
tgggcttaaaacccaccact

SEQ ID NO: 2 - DNA, 20 bp, synthetic construct
  source 1..20: mol_type = other DNA
cgggtttctgtgtaacgcat

SEQ ID NO: 3 - DNA, 32 bp, synthetic construct
  source 1..32: mol_type = other DNA
  misc_feature 32: note = T at position 32 is conjugated to fluorescein or other small organic dye, fluorescent protein (FP), or quantum dot (QD) that can be a FRET donor
ccccatggtgatagttccatttatgatgcaat

SEQ ID NO: 4 - DNA, 32 bp, synthetic construct
  source 1..32: mol_type = other DNA
  misc_feature 32: note = G at position 32 is labeled with phosphate or other label
  misc_feature 1: note = A at position 1 is conjugated to LC Red 610 or other small organic dye, fluorescent protein (FP), and/or quantum dot (QD) that is a FRET acceptor
atcagaatgtcccaaagctgaaagaacaactg

SEQ ID NO: 5 - DNA, 21 bp, synthetic construct
  source 1..21: mol_type = other DNA
tcgtcgtgttctttatggtgc

SEQ ID NO: 6 - DNA, 20 bp, synthetic construct
  source 1..20: mol_type = other DNA
  misc_feature 7: note = "y" is c or t/u
  misc_feature 8: note = "y" is c or t/u
ataacgyygtgcagcaggtc

SEQ ID NO: 7 - DNA, 35 bp, synthetic construct
  source 1..35: mol_type = other DNA
  misc_feature 35: note = A at position 35 is conjugated tois conjugated to fluorescein or other small organic dye, fluorescent protein (FP), or quantum dot (QD) that can be a FRET donor
ttcttgacatggtgtcatatattgccatatcacca

SEQ ID NO: 8 - DNA, 35 bp, synthetic construct
  source 1..35: mol_type = other DNA
  misc_feature 35: note = A at position 35 is labeled with phosphate or other label
  misc_feature 1: note = A at position 1 is conjugated to LC Red 610 or other small organic dye, fluorescent protein (FP), and/or quantum dot (QD) that is a FRET acceptor
agggtggaatttactcattacatcaccaacaatcc